(19) United States
(12) Patent Application Publication
(10) Pub. No.: US 2007/0218049 A1
(43) Pub. Date: Sep. 20, 2007

(54) Nanoparticle based photodynamic therapy and methods of making and using same

(76) Inventors: Wei Chen, Stillwater, OK (US); Jun Zhang, Xincheng District Huhhot (CN)

Correspondence Address: DUNLAP, CODDING & ROGERS P.C., PO BOX 16370, OKLAHOMA CITY, OK, 73113, US

(21) Appl. No.: 11/347,075

(22) Filed: Feb. 2, 2006

(51) Int. Cl. A61K 39/395; A61K 9/50

(52) U.S. Cl. 424/130.1; 424/499; 607/87

(57) ABSTRACT

A novel method for cancer treatment that combines radiotherapy and photodynamic therapy (PDT). More particularly, luminescent nanoparticles with attached photosensitizers, such as porphyrins, are used as a new type of agent for photodynamic therapy. Upon exposure to ionizing radiation, light will emit from the nanoparticles to activate the photosensitizers; as a consequence, a singlet oxygen is produced to augment the killing of cancer cells by ionizing radiation. No external light is necessary to activate the photosensitizing agent within tumors. The combination of radiotherapy and PDT is more efficient than either used alone.

The government may own certain rights in and to this application pursuant to: (i) a grant from the Department of Defense (Army Medical) A043-187-0258 (Contract No. W81XWH-05-C-0101).

All references to patent applications, issued patents, articles, trade journals and manuals are expressly intended to incorporate such materials expressly herein in their entirety as if set forth specifically herein. The above list of types of materials to be incorporated herein are only provided as examples and should not be regarded as limiting as to the type of materials expressly incorporated herein.

### BACKGROUND OF THE INVENTION

1. Field of Invention

Of the many diseases that threaten our lives, cancer ranks very high in terms of public fear. Cancer is a group of diseases characterized by the uncontrolled growth and spread of abnormal cells. If the spread of these abnormal cells is not controlled, it oftentimes will result in the death of the patient. The National Cancer Institute estimates that about 1,368,030 new cancer cases were expected to be diagnosed in 2004. Since 1990, more than 18 million new cancer cases have been diagnosed. In 2005, about 563,700 Americans are expected to die of cancer, more than 1,500 people a day. Cancer is the second leading cause of death in the USA, exceeded only by heart disease. In the US, cancer causes 1 out of every 4 deaths.

Much effort has been and continues to be dedicated to curing the various types of cancer. Actually, the prognosis for someone diagnosed with cancer is not as dire as is commonly believed. Many cancers, such as early stage cancer of the larynx, childhood leukemia and Hodgkin's disease, are highly curable. Early in their development, malignant tumors are generally well localized. In this case, a local treatment such as surgical excision or radiation therapy is indicated. If the tumor is inaccessible or is intimately entwined with a vital anatomic structure or if regional spread has occurred, surgery may not be a feasible treatment option. In these cases, radiation therapy and chemotherapy may have better outcomes.

The presently disclosed and claimed invention provides, in one specific embodiment, a novel and nonobvious method for the treatment of cancer that combines radiotherapy and photodynamic therapy. According to the presently disclosed and claimed invention, luminescent nanoparticles with attached photosensitizers, such as porphyrins, are used as an agent for photodynamic therapy. Upon exposure to ionizing radiation, light is emitted from the nanoparticles and thereby activates the photosensitizers; as a consequence, a singlet oxygen is produced which is capable of augmenting the killing of cancer cells by ionizing radiation. With this novel therapeutic approach, no external light is necessary to activate the photosensitizing agent within tumors. Thus, this new modality is termed a self-lighting photodynamic therapy or SLPDT for short. The combination of radiotherapy and photodynamic therapy provides a less expensive and more efficient treatment for cancer patients. In addition, such a methodology can also be used for the treatment of infectious diseases caused by viruses or bacteria as well as for the sterilization, neutralization or destruction of viruses, bacteria, chemical warfare or biological warfare agents.

2. Background of the Related Art

Currently, radiation therapy is still the most common and efficient treatment for cancers. In North America, more than half of all cancer patients during the course of their illness receive radiation therapy. Radiation works because it causes lethal damage to cells. In addition to primary damage from direct deposition of radiation into biologically vital macromolecules, secondary electrons from radiation create highly reactive radicals in the intracellular compartment; the result is that these radicals can chemically break bonds in cellular DNA and cause cells to lose their ability to reproduce. In order to damage DNA, the energy of radiation has to exceed a few tens of electron-volts. However, if the radiation is delivered from outside the body, as in teletherapy, then photon energies of several million electron-volts are needed to avoid deposition in superficial structures and reach the deeper sealed tumors in the body. By contrast, brachytherapy (Brachytherapy which involves the placement of radioactive sources either in tumors or near tumors in which the radiation is limited to short distances) implants can be successfully performed with radionucleotides that emit photons with energies as low as 20 keV, which is another reason why brachytherapy is becoming even more popular as a treatment modality. Both teletherapy and brachytherapy have been and are widely used for cancer treatment.

Photodynamic therapy has been designated as a “promising new modality in the treatment of cancer” since the early 1980s. This can be partly attributed to the very attractive basic concept of PDT—the combination of two therapeutic agents: a photosensitizing drug and light. Both the light and the photosensitizing agent are relatively harmless by themselves but, when combined in the presence of oxygen, can result in selective tumor destruction. The mechanisms of PDT have been investigated extensively. (see e.g., Macdonald and Dougherty, Journal of Porphyrins and Pthalocyanines, 2001, 5, 105)

In general, PDT agents produce singlet oxygen. Singlet oxygen is a highly reactive form of molecular oxygen that is produced by inverting the spin of one of the outermost electrons. Normally, the triplet ground state of oxygen has two unpaired electrons residing separately in the outermost anti-bonding orbitals. The extreme reactivity of singlet oxygen arises from the paring of the two electrons into one of the anti-bonding orbitals. Singlet oxygen is so reactive that it has a lifetime ranging from 10-100 μs in organic solvents. This restricts its activity to a spherical volume 10 nm in diameter centered at its point of production (Macdonald and Dougherty, 2001) which allows any cell destruction to be confined to a limited volume.

Photosensitized generation is a simple and controllable method for the production of singlet oxygen, requiring only oxygen, light of an appropriate wavelength, and a photosensitizer capable of absorbing and using the light energy to excite the oxygen to its single state. (Macdonald and Dougherty, 2001) In oxygenated environments, the photosensitizers readily transfer their energy to ground state molecular oxygen (³O₂) to produce singlet oxygen. The photosensitizer and oxygen interact through the triplet states because oxygen has a unique, triplet-ground state and low-lying excited states. The energy required for the triplet-to-singlet transition in oxygen is 22 kcal mol⁻¹, which corresponds to a wavelength of 1274 nm. (Macdonald and Dougherty, 2001) Thus, relatively low energy is needed to produce singlet oxygen.

PDT efficiency is largely determined by the production of singlet oxygen. Singlet oxygen production efficiency is determined by the photosensitizer, light (intensity and wavelength), and oxygen concentration. An ideal photosensitizer for in vivo PDT must be easy to deliver to tumors, water-soluble, readily available, and cost effective with no dark toxicity, mutagenicity, or carcinogenicity. Further, the activation of the photosensitizer must be initiated by an appropriate wavelength of light. To date, there are no photosensitizers that satisfy all these requirements. (Macdonald and Dougherty, 2001)

Typically, fluences of 50-500 J/cm² of red light are used in clinical PDT with Porphyrins. (Macdonald and Dougherty, 2001) The activating light is most often generated by lasers because they produce highly coherent monochromatic light. Because lasers are inconvenient for use in an operating room or clinic, light is usually produced away from the patient and delivered through fiber-optic cables to the treatment site, often through an endoscope. Similar to radiation brachytherapy for interstitial treatment, a diffusing fiber is inserted into the tumor to be treated. Another means of delivering light is to use an external light source producing wavelengths that will penetrate directly into the tissue. The depth of penetration depends upon the optical properties of the tissue and the wavelength of the light employed. When photons are directed at tissue, a portion is reflected by the surface and the rest scatters in the pores (either endogenous tissue chromophores or exogenous molecules such as the photosensitizer). Theoretically, a few photons may pass completely through the tissue volume, although this number is very small.

Wavelengths of less than 800 nm are scattered with increasing efficiency by macromolecules because they are equal to or less than the size of the particles. Therefore, it is advantageous to have photosensitizers that absorb near 800 nm to maximize the treatment depth in many different tissues. Furthermore, to increase the penetration into tissue, it is useful to move into the NIR spectral range (700-1100 nm), where most tissue chromophores, including oxyhemoglobin, deoxyhemoglobin, melanin, and fat, absorb weakly. However, most photosensitizers have absorption bands at wavelengths shorter than 800 nm.

Recently, US patent application publication No. 20020127224, was filed by James Chen on Sep. 12, 2002 and is entitled “Use of photoluminescence nanoparticles for photodynamic therapy.” According to the published application, without commenting on the accuracy of the application or the statements contained therein, this application purportedly discloses compositions and methods that can be used to effect a photodynamic therapy (PDT) such as cancer treatment or gene transcription. Disclosed compositions for use include light-emitting nanoparticles that absorb light of one wavelength emitted by a light source and emit light of another wavelength that activates a PDT drug. Light-emitting nanoparticles include quantum dots, nanocrystals, and quantum rods as well as mixtures of these nanoparticles. The nanoparticles may be delivered to a patient in a liquid carrier or as part of a solid carrier such as a biocompatible polymeric film, a polymeric sheath, or other carrier suitable for introduction at the site to be treated. In one embodiment of the invention, light-emitting nanoparticles are localized at the treatment site by either joining them to the PDT drug covalently or non-covalently through linkage groups such as biotin/avidin, or the nanoparticles are localized at the treatment site by attaching the nanoparticles to a linkage group that has affinity for e.g., cells or proteins produced at the site to the treated. A sufficient number of light-emitting nanoparticles are delivered to the treatment site to activate the PDT drug and effect treatment.

Notably, however, this published patent application by Chen is insufficient to meet all of the objectives of the presently disclosed invention. Namely, the Chen patent application: (1) does not teach scintillation luminescence and scintillation nanoparticles; (2) does not teach long afterglow or persistent luminescence nanoparticles; (3) does not teach size and dielectric doubly confined nanoparticles; (4) does not teach the combination of radiotherapy and PDT or hyperthermia with PDT; (5) does not teach doped nanoparticles; and (6) does not teach thermoluminescence and its use as a PDT light source.

In order to solve these above-described problems, a new PDT agent system in which the light is generated from luminescent nanoparticles attached to the photosensitizers is disclosed herein. The presently disclosed PDT approach or method is shown generally in the schematic of FIG. 1. Photosensitizers, such as porphyrins, are coated or attached to the luminescence nanoparticles, particularly scintillation luminescence and persistent luminescence nanoparticles. Upon excitation with radiation beams such as X-rays, light is generated from the nanoparticles and activates the photosensitizers to produce singlet oxygen for PDT.

### SUMMARY OF THE INVENTION

The present invention relates, in general, to a method for photodynamic therapy. In one embodiment, this method includes the steps of (1) providing at least one luminescent nanoparticle; (2) providing at least one photosensitizer that is functionally associated with the at least one luminescent nanoparticle; and (3) providing an excitation source. The excitation source, in this embodiment, is capable of exciting the at least one luminescent nanoparticle and thereby exciting the at least one photosensitizer to provide the photodynamic therapy.

The luminescence of the at least one luminescent nanoparticle is selected from the group consisting of scintillation luminescence, persistent luminescence, afterglow, thermoluminescence, magnetoluminescence, phosphorescence, photostimulated luminescence, and bioluminescence. Furthermore, the at least one luminescent nanoparticle may be selected from the group consisting of semiconductor nanoparticles, insulator nanoparticles, doped nanoparticles, ceramic nanoparticles, metallic nanoparticles, organic nanoparticles, inorganic nanoparticles, core-shell nanoparticles, size confined nanoparticles, dielectric confined nanoparticles, size and dielectric doubly confined nanoparticles, and combinations thereof.

In another embodiment, the at least one luminescent nanoparticle has a diameter from about 0.1 nm to about 5000 nm and may be selected from the group consisting of CaF₂:Mn²⁺, CaF₂:Eu²⁺, CaF₂:Ce³⁺, BaFBr:Eu²⁺, BaFBr:Mn²⁺, CaPO₄:Mn²⁺, ZnS, CaPO₄:Eu²⁺, ZnO, CdS, CdSe, CdTe, TiO₂ nanoparticles and combinations thereof.

In an additional embodiment (or as part of the previous or later embodiments) the at least one photosensitizer may be selected from the group consisting of organic dyes, porphyrins and their derivatives, flavins, organometallic species, inorganic compounds, fullerenes, semiconductor nanoparticle photosensitizers, and combinations thereof. In particular, the at least one photosensitizer may be selected from the group consisting of haematoporphyrin, verteporfin, tetrakis(o-aminophenyl)porphyrin, and combinations thereof. Alternatively, the at least one photosensitizer may be selected from the group consisting of ZnO nanoparticles, Si nanoparticles, TiO₂ nanoparticles and combinations thereof.

The at least one nanoparticle and the at least one photosensitizer may be operably associated with one another by a functional ligand such as cysteine. Alternatively, the at least one nanoparticle and the at least one photosensitizer may be operably associated with one another by electrostatic interaction. Thirdly, the at least one nanoparticle and the photosensitizer may be operably associated with one another by coating the at least one photosensitizer on the surface of the at least one nanoparticle.

In one embodiment, the at least one photosensitizer may be selected from the group consisting of TiO₂, ZnO and combinations thereof and the at least one nanoparticle may be selected from the group consisting of CaF₂:Eu²⁺, ZnO and combinations thereof.

In all embodiments the excitation source may be an ionizing radiation source such as X-rays, alpha-particles, beta-particles, neutrons, gamma rays and combinations thereof. Alternatively, the radiation source may be at least one radioactive atom doped in or bound to the at least one luminescent nanoparticle. Further, the radiation source may be capable of at least two functions (1) radiation therapy and (2) excitation of the at least one luminescent nanoparticle. When this occurs, the excited luminescent nanoparticle is capable of exciting the at least one photosensitizer to provide photodynamic therapy.

In all embodiments, the excitation source may also be heat, whether such heat is generated by infrared light, a magnetic field and combinations thereof.

Generally, the presently disclosed and claimed invention(s) may preferentially be used for the photodynamic treatment of cancer or a tumor in a patient such as a bladder tumor or breast cancer, prostate cancer, skin cancer, ovarian cancer and combinations thereof. Additionally, the presently disclosed and claimed invention(s) may also be used for the photodynamic treatment of an infectious disease in a patient such as one caused by a bacteria or a virus. In such cases, the bacteria or virus may be E. coli, an influenza virus, a severe acute respiratory syndrome (SARS) virus and combinations thereof.

In one further embodiment, the presently disclosed and claimed invention(s) may further include the step of providing targeting of the least one luminescent nanoparticle that is functionally associated with the at least one photosensitizer. Such targeting may be provided by a method selected from the group consisting of antibody-antigen targeting, receptor targeting and combinations thereof. Particularly, and in one specific embodiment, the targeting is provided by the conjugation of folic acid to the at least one luminescent nanoparticle. In yet another specific embodiment, the targeting is provided by the encapsulation of the luminescent nanoparticle that is functionally associated with the photosensitizer in at least one liposome, wherein said liposome has a functionalized surface that acts as a receptor.

Generally, the presently claimed and disclosed invention(s) also provide for a method for photodynamic therapy. In this embodiment, the method includes the steps of (1) providing at least one luminescent photosensitizer nanoparticle, and (2) providing an ionizing radiation source. In this embodiment, the ionizing radiation source is capable of exciting the at least one luminescent photosensitizer nanoparticle to provide photodynamic therapy. In this embodiment, the at least one luminescent photosensitizer nanoparticle is selected from the group consisting of ZnO nanoparticles, Si nanoparticles, TiO₂ nanoparticles and combinations thereof.

### BRIEF DESCRIPTION OF THE SEVERAL VIEWS OF THE DRAWINGS

FIG. 1 is an illustration showing (a) the molecular structure of a porphyrin at the top of the page and (b) at the bottom, porphyrins are shown linked to luminescent nanoparticles through L-cysteine. The thiol groups bond with the nanoparticles and the amine groups or the carboxylic groups or both of the groups bond with the porphyrins. The porphyrins can be activated by the light from the nanoparticles as a result of energy transfer.

FIG. 2 shows A) the emission spectrum of BaFBr:Eu²⁺, Mn²⁺ nanoparticles excited by X-ray, and B) the absorption spectrum of Haematoporphyrin. The emission spectrum overlaps with the absorption spectrum of the porphyrin which facilitates energy transfer from the nanoparticles to the porphyrins.

FIG. 3 shows the emission spectra of differently sized CdSe nanoparticles that can be used as the light sources for PDT conjugated agents.

FIG. 4 shows the X-ray excited luminescence spectra of CaF₂:Mn²⁺ nanoparticles at different total irradiation times (i.e. different doses).

FIG. 5 shows the X-ray excited luminescence spectrum of CaF₂:Eu²⁺ nanoparticles.

FIG. 6 is a schematic model for persistent luminescence (PL) or afterglow with multiple trapping energy levels (T1, T2 and T3). VB=Valence Band, CB=Conduction Band.

FIG. 7 shows the afterglow spectrum of CaF₂:Mn²⁺ nanoparticles 20 minutes after 1 minute of X-ray irradiation.

FIG. 8 shows the afterglow images of BaFBr:Eu²⁺, Mn²⁺ nanophosphor at 2, 4 and 8 minutes after the X-ray excitation was turned off. The afterglow lasted for two hours

FIG. 9 shows the emission spectrum of ZnO nanoparticles (solid) and absorption spectrum of porphyrin (dash).

FIG. 10 shows the excitation spectrum of the porphyrin TOAP (solid) and the emission spectrum of CdS nanoparticles (dash). The absorption of TOAP overlaps with the emission of CdS nanoparticles.

FIG. 11 is a schematic illustration of the chemical conjugation of tetrakis(o-aminophenyl)porphyrin (TOAP) to a nanoparticle through cysteine. For simplicity, only one TOAP molecule is shown conjugated to a nanoparticle. Abbreviations: NP, nanoparticle; TOAP, tetrakis(o-aminophenyl)porphyrin; EDC, 1-(3-Dimethylaminopropyl)-3-ethylcarbodiimide; NHS, N-hydroxysuccinimide.

FIG. 12 shows the emission spectra of CaF₂:Eu²⁺ nanoparticles (solid) and the CaF₂:Eu²⁺ nanoparticle/porphyrin system (dash) excited at 318 nm.

FIG. 13 shows the fluorescence emission spectra of porphyrin (solid), CdS nanoparticles (dash) and nanoparticle/porphyrin conjugates (dot). The excitation wavelength is 396 nm.

FIG. 14 shows the emission spectra of ZnO nanoparticles (solid), porphyrin (dash) and ZnO/porphyrin conjugates (dot).

FIG. 15 shows the fluorescence decay of the CaF₂:Eu²⁺ nanoparticle/porphyrin system excited at 318 nm with emission at 420 nm.

FIG. 16 shows the luminescence change of 1,3-diphenylisobenzofuran (DPBF) in a porphyrin system with respect to the duration of the exposure to light. The luminescence of DPBF is quenched by singlet oxygen and the decrease of the intensity can be used to estimate the amount of singlet oxygen generated. A is the emission spectra of DPBF at different duration of time and B is the change of the peak intensity with duration of time.

FIG. 17 is a schematic illustration of a chemical process for making TOAP/nanoparticle/folic acid conjugates.

FIG. 18 is a schematic illustration of linking TOAP and folic acid to a luminescent nanoparticle. For simplicity, only one TOAP and one folic acid molecule per nanoparticle is shown. The carboxylic group of the cysteine is used to connect with amino group of TOAP, while the amino group of the cysteine is linked to the carboxylic group of the folic acid by an amide bond.

FIG. 19 shows the encapsulation of nanoparticle PDT agents into a liposome via an opening-and-closing process that is used for drug delivery.

### DETAILED DESCRIPTION OF THE INVENTION

Before explaining at least one embodiment of the invention in detail, it is to be understood that the invention is not limited in its application to the details of construction and the arrangements of the components set forth in the following description or illustrated in the drawings. The invention is capable of other embodiments or of being practiced or carried out in various ways. Also, it is to be understood that the phraseology and terminology employed herein is for purpose of description and should not be regarded as limiting.

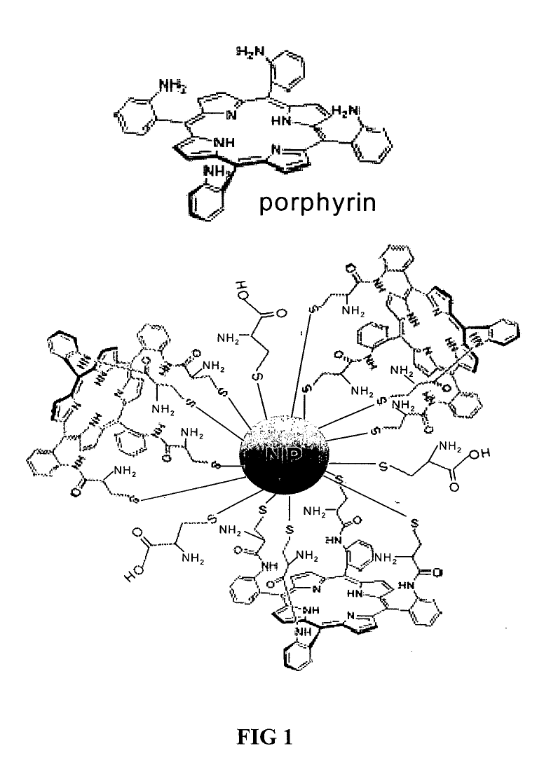

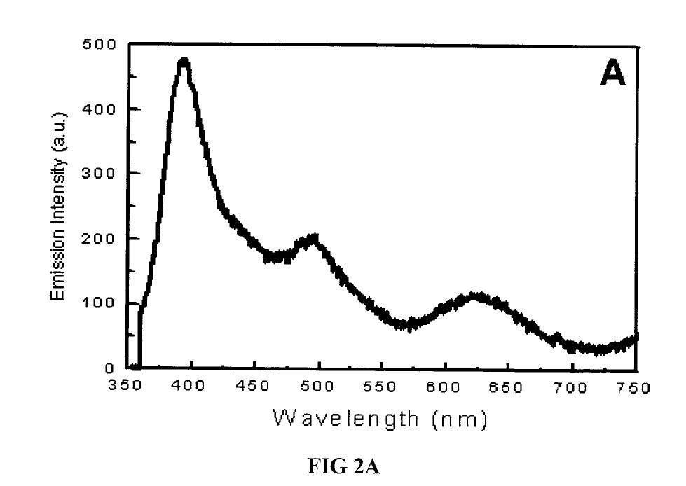

The presently disclosed and claimed PDT approach or method is shown schematically in FIG. 1. Photosensitizers such as porphyrins are coated or attached to scintillation nanoparticles. Upon excitation by ionizing radiation such as X-rays, light is generated from the nanoparticles and activates the photosensitizers to produce singlet oxygen for PDT. The advantages of this PDT system are as follows: (1) No external light is necessary; (2) Two effective treatments are combined; (3) The treatment is simple, economical, and highly efficient; and (4) Some of the many binding sites of the nanoparticles can be used for binding targeting molecules such as folic acid. Many organic dyes, flavins, porphyrins and their derivatives, and other biomolecules are efficient photosensitizers (PSs).

Photosensitized generation is a simple and controllable method for the production of singlet oxygen, requiring only oxygen, light of an appropriate wavelength, and a photosensitizer capable of absorbing and using the light energy to excite oxygen to its single state. (Macdonald and Dougherty, 2001) In oxygenated environments, the photosensitizers readily transfer their energy to ground state molecular oxygen (³O₂) to produce singlet oxygen. The photosensitizer and oxygen interact through the triplet states because oxygen has a unique, triplet-ground state and low-lying excited states. The energy required for the triplet-to-singlet transition in oxygen is 22 kcal mol⁻¹, which corresponds to a wavelength of 1274 nm. Thus, relatively low energy is needed to produce singlet oxygen. As discussed above singlet oxygen augments the ability of radiation therapy to kill cancerous cells.

Nanoparticles are useful as PS carriers for use in the presently disclosed and claimed invention because (1) they can be made hydrophilic; (2) they possess enormous surface area, and their surface can be modified with functional groups possessing a diverse array of chemical or biochemical properties; (3) owing to their sub-cellular and nanometer size, they can penetrate deep into tissue through fine capillaries, cross the fenestration, and present themselves in the epithelial lining so that they are generally taken up efficiently by cells; (4) they can be economically and efficiently made: numerous strategies for the preparation of nanomaterials exist; and (5) once made, they can be covalently grafted with a variety of molecules. Most importantly, the inventive application of luminescent nanoparticles disclosed and claimed herein provides not only carriers for delivery of photosensitizers but also provides a potentially activatable light source directly to the targeted tissue.

Recently, it has been demonstrated that some nanostructured materials can be photoactivated to produce singlet oxygen. Fullerenes are three-dimensional, hollow, cage-like molecules composed of hexagonal and pentagonal groups of atoms, usually, but not always, carbon atoms. Fullerenes are good candidates for PDT and medical applications. The efficiency of singlet oxygen formation for C₆₀ (unity at 532 nm) is the highest among all photosensitizers investigated to date. (see e.g., J. W. Arbogast, A. P. Darmanyan et al, Journal of Physical Chemistry 1991, 95: 11) The efficient generation of singlet oxygen by photoexcited C₆₀ and C₇₀ makes fullerenes useful candidates for PDT. However, fullerenes absorb strongly in the UV and moderately in the visible region. The main drawback for the application of fullerenes in PDT is their poor absorption in the red region of the visible spectrum. The absorption spectrum of C₇₀ is somewhat red-shifted compared with that of C₆₀ but the absorbance of C₇₀ beyond 700 nm is still very low. This is one of the drawbacks for their application in PDT because UV and visible light are difficult to deliver into deep tissue. Once the problem of light delivery in UV/visible regions is solved, the application of C₆₀ and C₇₀ in PDT is tremendous, and one aspect of the presently disclosed invention is the use of scintillation nanoparticles attached to photosensitizers, where the scintillation nanoparticles act as light sources.

Recently, it was reported that semiconductor nanoparticles such as TiO₂ and ZnO are effective photocatalysts that are capable of generating singlet oxygen for killing cancer cells and bacteria. [Wang et al, Journal of Materials Chemistry, 2004, 14: 487] For example, it was reported by Xu et al that B-chelated TiO₂ nanocomposite has a high efficiency of singlet oxygen generation when irradiated with visible light [Xu et al, Journal of Photochemistry and Photobiology B: Biology, 2002]. Similar to other photosensitizers, semiconductor nanoparticles such as TiO₂ and ZnO only have strong absorption in UV or visible ranges, which limits their application in PDT. Once again, the presently disclosed methodology provides a means of circumventing the problem of light activation. In particular, the use of Porphyrin, C₆₀, and TiO₂ nanoparticles as singlet oxygen generators in the presence of scintillation nanoparticles is disclosed and claimed—i.e., scintillation nanoparticles can and do act as efficient light sources for PDT activation.

Nanoparticle Selection for Self-Lighting

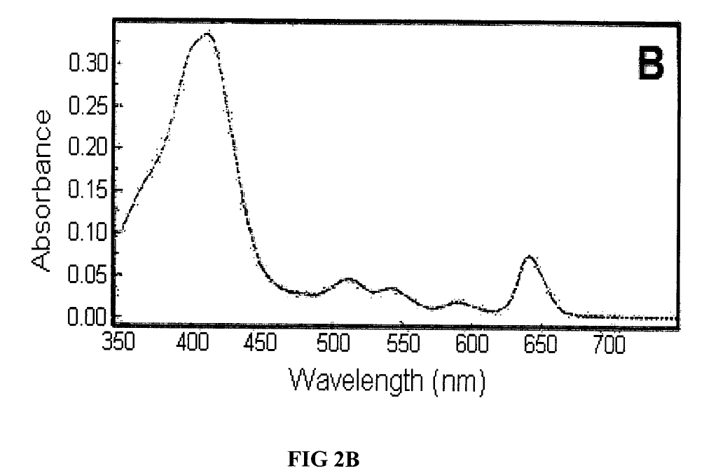

One of ordinary skill in the art would appreciate that there are numerous nanoparticle compositions that could be used in the presently described and claimed invention. In general, however, preferred nanoparticles for use with the present invention can be classified according to functional requirements and/or properties. In a preferred embodiment, by way of example but not to be considered limiting, nanoparticles selected for the presently disclosed and claimed inventive system should meet the following requirements: (1) The nanoparticle emission spectrum should match the photosensitizer's absorption spectrum. For example, if Porphyrin is used, the nanoparticle should have an emission at around 400 nm to match the absorption band of Porphyrins (see FIG. 2). Matching the nanoparticle emission with the photosensitizer absorption allows efficient activation of the photosensitizers and production of singlet oxygen; (2) The nanoparticles should have high luminescence efficiency, particularly under radiation by X-rays; (3) The particles should be non-toxic, water-soluble, and stable in biological environments; and (4) The particles should be easily attached to or linked with photosensitizers.

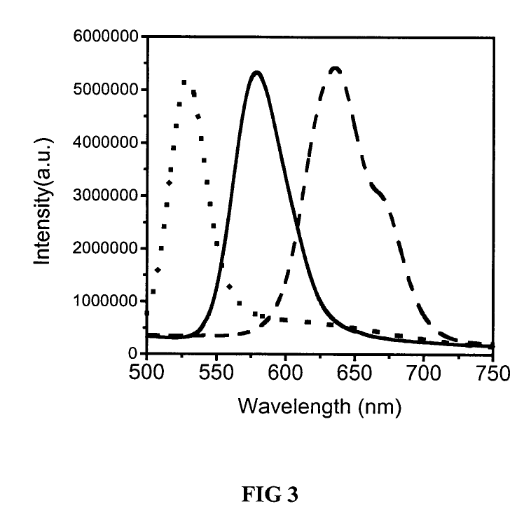

Nanoparticles have higher luminescence quantum efficiency than conventional phosphors. This is due to the large increase in electron-hole overlap, thus yielding an increase in the oscillator strength and, as a consequence, enhanced luminescence quantum efficiency. Further, the emission energy or wavelength is adjustable by nanoparticle size (see FIG. 3). In addition, different emission wavelengths or energies can be obtained by using different dopants. Therefore, in the presently disclosed invention, we can control the particle emission wavelength to match the absorption band of the photosensitizers by controlling the particle size or using different dopants.

It is worth noting that many different names have been used for nanoparticles, including nanocrystals, nanocrystalline particles, clusters, nanoclusters, quantum dots, dots, low-dimensional materials, nanorods, nanowires, and nanostructures. Herein such terms should be considered interchangeable and non-limiting. In a preferred embodiment, the presently disclosed and claimed invention incorporates nanoparticles that can be defined as particles having geometric dimensions between from about 0.1 nm to about 5000 nm. These preferred nanoparticles may be spherical or asymmetric.

Scintillation Luminescence Nanoparticles

In yet another embodiment of the presently disclosed and claimed invention, high scintillation luminescence (SL) of a certain wavelength is produced from the nanoparticles excited by ionizing radiation such as X-rays. High-density materials have high stopping power and a high absorption coefficient for radiation; thus, phosphors made with high atomic number elements will have high scintillation luminescence efficiency. Also, if the Stokes shift is smaller, the luminescence efficiency is higher. Theoretically, this is related to electron-phonon coupling. If electron-phonon coupling is weak, then the Stokes shift is small, and the scintillation luminescence efficiency will be higher. Electron-phonon coupling is weaker for smaller sized nanoparticles as a result of the decreased density of states. Consequently, the Stokes shift is less for smaller sized particles. These factors indicate that SL can be enhanced in nanoparticles through quantum size confinement.

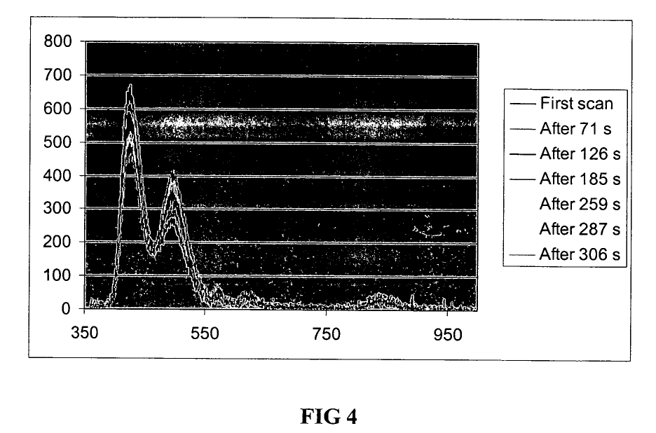

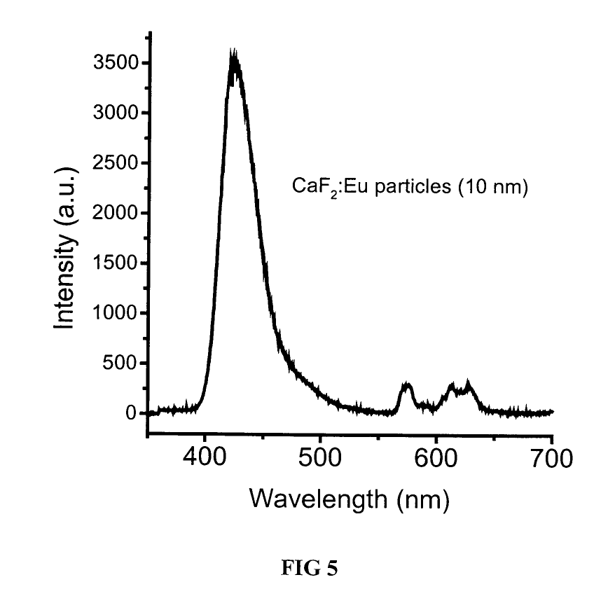

Based on the above description and teachings, and considering the luminescence wavelength (for matching PS absorption), efficiency and toxicity, CaF₂, BaFBr, CaPO₄, ZnO, and ZnS doped nanoparticles were initially chosen and tested as light sources for the presently disclosed and claimed SLPDT system. The emission spectra of these nanoparticles can be matched perfectly to the absorption spectra of Porphyrins, fullerenes, and TiO₂ nanoparticles. For example, FIG. 4 displays the emission spectrum of CaF₂:Mn²⁺ particles excited by X-ray. The particles have two emission bands, one peaking at around 400 nm and the other at 540 nm. The emission spectrum of the CaF₂:Mn²⁺ nanoparticle matches perfectly the absorption spectra of most Porphyrins. Similarly, the emission spectrum of CaF₂:Eu²⁺ nanoparticles is overlapped with the absorption spectra of most porphyrins and can be used for the presently disclosed and claimed SLPDT system (see FIG. 5). Note that in addition to x-ray radiation, neutrons or alpha, beta, or gamma radiation could also be used to cause luminescence from the nanoparticles.

Long-Afterglow Nanoparticles

Persistent or long-afterglow phosphors are luminescent materials with long decay lifetimes, ranging from a few minutes to tens of hours. These materials can be widely used for applications such as road signs, billboards, graphic arts, interior decoration, and emergency lighting. The presently disclosed and claimed invention also combines scintillation and afterglow luminescence for use with the SLPDT system. Afterglow and scintillation luminescence are closely related. The luminescence during X-Ray irradiation is called scintillation luminescence, while the luminescence after the X-ray irradiation is tuned off is called afterglow. Generally, an afterglow nanoparticle also has scintillation luminescence; however, a scintillation nanoparticle might not have afterglow. Nanoparticles that exhibit both scintillation and afterglow luminescence can also be used with the presently disclosed and claimed SLPDT system. When such “afterglow” nanoparticles are used in the SLPDT system, the radiation dose can be greatly reduced. For example, if scintillation nanoparticles without afterglow are used, 30 seconds of radiation dosing may have to be used to generate enough photons for PDT activation; whereas, if scintillation nanoparticles with afterglow are used, only 10 seconds of radiation dosing is needed to generate enough photons for PDT because extra photons are contributed from the afterglow. Therefore, the benefits and applications of nanoparticles having afterglow are tremendous.

Afterglow is quite similar to the X-ray induced photostimulated luminescence (PSL) (Chen, 1998). Both are based on the use of lattice defects for storing the excitation energy. The only difference is that light is used for stimulation in PSL, but ambient temperature ‘heat’ is used for thermal stimulation of afterglow luminescence at room temperature.

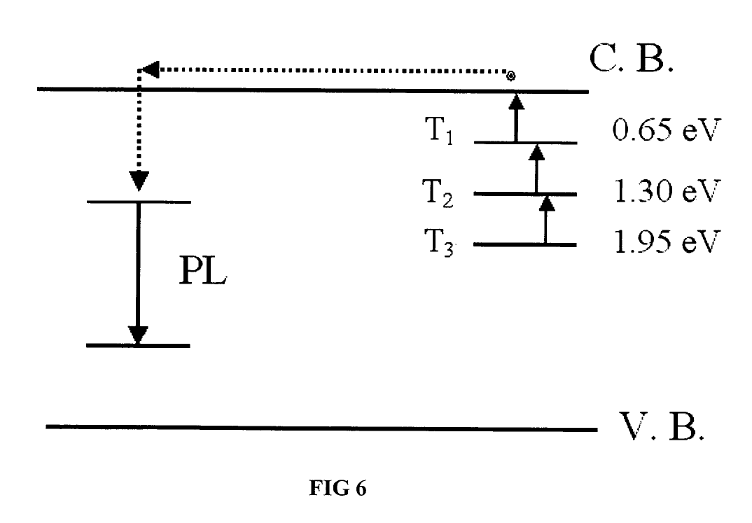

In order to design efficient afterglow nanoparticles, it is crucial to have trapping levels located at a suitable energy level in relation to the thermal release rate at room temperature. It is known that a trap depth of about 0.65 eV is optimal for persistent luminescence at room temperature. Materials with many trapping levels with energy separation between each pair of about 0.65 eV have been designed and developed (See FIG. 6). In this case, the ‘heat’ at room temperature can relax the energy stored at the trap level T1 but not at deep levels T2 and T3, while the energy stored at T2 can thermally migrate to T1. The same is true for levels T3 and T2. Thus, we can store much more energy can be stored in the phosphors, and the energy can be slowly released at room temperature.

We should point out that the temperature in vivo is a little higher than room temperature (298 K). So the ‘heat’ energy for in vivo is actually higher than the ‘heat’ energy at room temperature. This actually favors the afterglow luminescence for thermal stimulation.

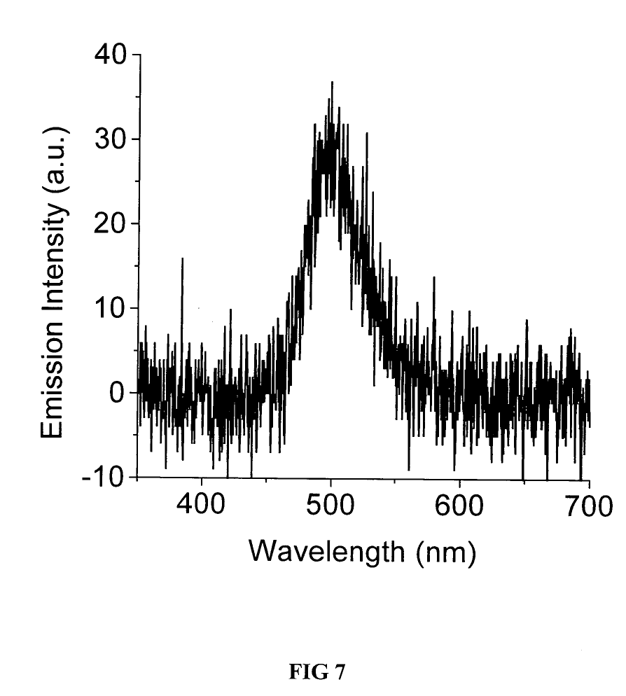

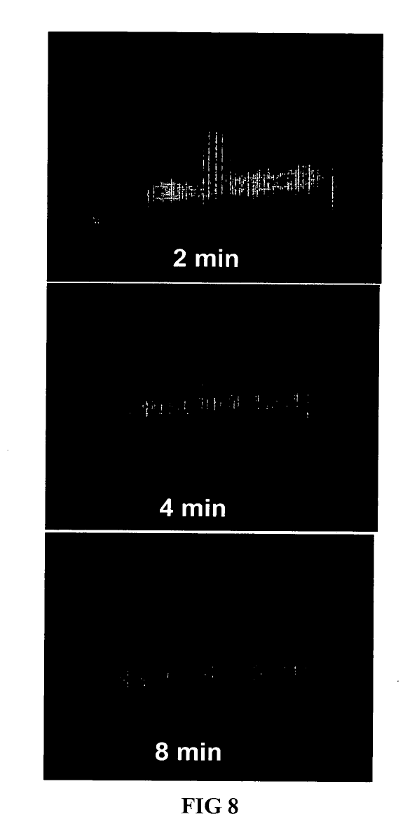

Host materials with many trapping levels as shown in FIG. 6 are necessary components of the afterglow embodiment of the presently disclosed and claimed SLPDT system. Through investigation, materials with three trapping levels have been designed and fabricated. The results show that the material has trapping levels with depths of 2.25, 1.65, 1.45, and 0.8 eV. The energy separations between each set of two neighboring levels are 0.60, 0.20, and 0.65 eV, respectively. Electrons (energy) can migrate from the deep levels to the low levels (Chen, 2003). This indicates that these materials can be used as long-lasting afterglow nanoparticles. FIG. 7 shows the afterglow spectrum of CaF₂:Mn²⁺ nanoparticles 20 minutes after 1 minute of X-ray irradiation and FIG. 8 display the afterglow images of BaFBr:Eu²⁺, Mn²⁺ nanophosphor at 2, 4 and 8 minutes after the X-ray excitation was turned off. The afterglow lasted for two hours. Afterglow nanoparticles have also been designed and fabricated that can last up to 2 hours, and the afterglow longevity can be tuned and controlled to meet the need for the presently disclosed and claimed SLPDT system.

X-Ray Induced Photostimulated Luminescence and Thermally Stimulated Luminescence

As mentioned hereinabove, x-ray radiation can lead to trapped states. Traps that are deep enough to prevent relaxation at room temperature, can still be relaxed by higher temperatures (thermally stimulated luminescence, TSL) or light of the appropriate energy (photostimulated luminescence, PSL). For PSL, usually red or near infrared light is used to relax a trapped state which then emits UV or blue light. The energy of the emitted light is greater than the energy of the stimulating light. The extra energy comes from the recombination of the trapped electron or hole, unlike in two-photon absorption where the absorption of two photons of low energy are required for the emission of one photon of higher energy.

TSL and/or PSL may be used to release the trapped energy from nanoparticles after an ionizing radiation treatment. This would require an additional application of heat or light. Localized heating can be provided by infrared light or magnetic fields or other methods used for hyperthermia.

Size and Dielectric Doubly Confined Nanoparticles

The luminescence color and quantum yield of a nanoparticle is closely related to its size. Quantum size confinement not only yields an increase in the energy band gap and the splitting of the electronic states, but also changes the density of states. In the case of nanoparticles, the density of states becomes more discrete as dimensionality decreases, and large optical absorption coefficients have been observed, which are favorable for luminescence. The luminescence efficiency is determined by the oscillator strength of the exciton. In nanostructured materials, the electron-hole overlap factor increases greatly due to quantum size confinement, thus yielding an increase in the oscillator strength. The oscillator strength is also related to the electron-hole exchange interaction that plays a key role in determining the exciton recombination rate. In bulk semiconductors, due to the extremely delocalized nature of electrons and holes, the electron-hole exchange interaction term is very small, while in molecule-size nanoparticles, due to confinement, the exchange term is very large. Therefore, there is a large enhancement of the oscillator strength for nanostructured materials. This is why nanoparticles have shown higher luminescence efficiencies than corresponding bulk materials, as reported for many semiconductor nanoparticles.

Nanoparticle luminescence can be further enhanced by dielectric confinement. If the dielectric constant (ε) of the nanoparticles is greater than that of the surrounding matrix, the electric force lines of the particles will penetrate into the matrix, and the Coulomb interaction will be enhanced. As a consequence, the binding energy and the oscillator strength of the exciton are greatly increased. This is called dielectric confinement (Takagahara, 1993). This effect can be used to further enhance the luminescence efficiency and stability of the nanoparticles. The effects of dielectric confinement on exciton stability, luminescence efficiency and decay lifetime have been investigated elsewhere in the literature. All the reported data show that improved luminescence efficiency and exciton binding energy are obtained by surrounding the nanoparticles with materials having lower dielectric constants than the nanoparticles.

ZnO (ε=1.7) and SiO₂ (ε=3.9) are good materials as their dielectric constants are lower than the CdS (ε=9.12), ZnS (ε=8.2), CaF₂ (ε=6.76), BaFBr (ε=14.17), and CaPO₄ (ε=14.5) nanoparticles. Thus, when these nanoparticles are coated with ZnO or SiO₂ to form core/shell structures, they have very high luminescence quantum efficiencies as a result of quantum size confinement and dielectric confinement. In addition, coating with ZnO or SiO₂ can increase the stability and reduce the toxicity of the nanoparticles. For example, coating CaF₂:Eu²⁺ nanoparticles with SiO₂ prevents the oxidation of Eu²⁺ to Eu³⁺ by singlet oxygen. This will not only protect the nanoparticles but also improves the SLPDT system's efficiency because the coating prevents the trapping of singlet oxygen by Eu²⁺ ions. The coating of CdS nanoparticles with SiO₂ or ZnO also reduces their toxicity because the coating prevents the leaking of Cd²⁺, which is toxic. However, the coating layer (shell) should be thinner than the energy transfer critical distance (˜10 nm) so that it does not block the energy transfer from the nanoparticles to the photosensitizers.

Suitability for Biological Applications

The selected nanoparticles, described hereinabove, possess the characteristics necessary for biological applications in that they are water-soluble and stable in biological environments. Most of these nanoparticles are benign and biologically inert. Calcium phosphate nanoparticles are non-toxic and biocompatible and are being developed as a vaccine adjuvant and for targeted gene delivery. They have been approved for human use in several European countries. Doping of Eu²⁺ or Mn²⁺ into CaPO₄ nanoparticles is easily accomplished because the radius of Ca²⁺ is close to that of Mn²⁺ and Eu²⁺, which also have the same valence state. CaF₂, ZnS, and ZnO are also biologically innocuous materials. For some other nanoparticles with a certain toxicity, such as CdTe and CdSe nanoparticles, the nanoparticles can be surface coated with benign materials such as silica, alumina, titanium oxide or polymers in order to reduce their toxicity.

Photosensitizer Selection for Photodynamic Therapy

For efficient treatment, it is important to apply efficient photosensitizers for singlet oxygen generation. Actually, many organic dyes, porphyrins and their derivatives, flavins, and organometallic species such as bis-cyclometallated Ir(III) complexes are efficient photosensitizers (PSs) and can be used for the presently disclosed and claimed SLPDT system. The photosensitizers currently approved by the FDA for PDT are Photofyrin (actually a mixture of porphyrins, including photoporphyrin, haematoporphyrin, hydroxyethyldeuteropophyrin); and verteporfin, a benzoporphyrin. These photosensitizers can also be used in the presently disclosed and claimed SLPDT system.

Fullerenes are good candidates for PDT and medical applications. The efficient generation of singlet oxygen by photoexcited C₆₀ and C₇₀ makes fullerenes potentially useful candidates for PDT. However, fullerenes absorb strongly in the UV and moderately in the visible region. The main drawback for the application of fullerenes in PDT is their poor absorption in the red region of the visible spectrum. The absorption spectrum of C₇₀ is somewhat red-shifted compared with that of C₆₀ but the absorbance of C₇₀ beyond 700 nm is still very low. This is one of the drawbacks for their application in PDT because UV and visible light are difficult to deliver into deep tissue. Once the problem of light delivery in UV/visible regions is solved, the application of C₆₀ and C₇₀ in PDT is tremendous. So, fullerenes and their derivatives are also considered as photosensitizers for use in the presently disclosed and claimed SLPDT system.

Nanophase Semiconductor Photosensitizers for Photodynamic Therapy

Recently, it has been demonstrated that some nanostructured materials can be photoactivated to produce singlet oxygen (Wang et al, 2004). Nanoparticle photosensitizers have some advantages that can overcome the shortcomings of organic photosensitizers as new and complimentary photosensitizers that can also be used in the presently disclosed and claimed SLPDT system. The main advantages of nanoparticle-photosensitizers are:
1. They can be made hydrophilic.
2. They possess relatively large surface area, and their surfaces can be modified with functional groups possessing a diverse array of chemical or biochemical properties.
3. Owing to their sub-cellular and nanometer size, nanoparticles can penetrate deep into tissue through fine capillaries and pass through the fenestrae into the epithelial lining so that they can be taken up efficiently by cells.
4. Nanoparticles have higher extinction or absorption coefficients than organic dyes.
5. Nanoparticles are more photostable than organic dyes for in vivo applications.

In semiconductor nanoparticles, due to quantum size confinement, the overlap of electron and hole wavefunctions are much higher than in bulk semiconductor materials. For the same reason, the absorption and luminescence efficiencies are stronger than for most organic dyes. In our measurements, we found that the absorption and emission of CdTe and CdS nanoparticles are one order of magnitude stronger than that of tetrakis(o-aminophenyl)porphyrin at roughly the same concentration. This indicates that such nanoparticles may be used as PDT agents. Semiconductor nanoparticles such as ZnO, TiO₂, Si, and CdSe have been reported to have photosensitizing properties (Wang et al, 2004). Among them, TiO₂ nanoparticles are believed to be the most promising and are being investigated for methods to kill cancer cells and bacteria (Xu et al, 2002).

Upon absorption of light, sensitizer molecules are excited to a short-lived singlet state. Following excitation, fast radiationless relaxation to the lower-lying triplet states occurs via intersystem crossing and ultimately yields the first excited triplet state T1 in a spin-allowed process. The longer the decay lifetime of the triplet state, the more time the photosensitizer has to act on the tumor tissue and to initiate biochemical and biophysical mechanisms, which cause tumor necrosis. The effectiveness of PDT treatment is closely related to the number of photons absorbed by the photosensitizer per unit volume of tissue. Tumor necrosis can only occur when the number of absorbed photons exceeds a so-called damage threshold. The triplet state lifetime limits the time available for a collision-induced transfer of the triplet state excitation energy to molecular oxygen or the other cellular compounds. Therefore a long triplet lifetime (>500 ns) is considered a precondition for efficient photosensitization (Macdonald and Dougherty, 2001).

The energy structure of semiconductor nanoparticles enables their photosensitizing potential. Similar to organic compounds, semiconductor nanoparticles also have singlet and triplet states, and the triplet state has slower decay than the singlet state. Furthermore, for semiconductor nanoparticles, a very long-lived state called dark exciton is formed. The dark exciton state is an optically forbidden excited state with a very weak emission and long decay lifetime of microseconds (μs). Once the excitation energy is relaxed to this dark exciton state, a long time is available for interaction with tumor tissue, which enhances the PDT efficiency. In addition, surface states are commonly observed in semiconductor nanoparticles due to their large surface-to-volume ratio. Based on our data, the decay lifetime of surface states is about 20 times longer than the exciton decay lifetime. All these data indicate that semiconductor nanoparticles are potentially efficient PDT agents deserving more investigation.

The TiO₂ nanoparticle is so far the most efficient PDT agent among nanoparticles, and it has been widely investigated as a modality for cancer treatment and bacterial sterilization (Wang et al 2004). TiO₂ is a biocompatible material, which encourages the application of TiO₂ nanoparticles as a PDT agent for cancer treatment. However, the energy gap of TiO₂ is about 3.5 eV, and the absorption peaks of TiO₂ nanoparticles are in the blue and ultraviolet ranges, which hinders the treatment of deep tumor tissue. In order to solve this drawback, scintillation nanoparticles are used as light sources for TiO₂ nanoparticle PDT agents, similar to those designed for porphyrins described elsewhere herein.

Bioconjugation of Nanoparticles and Photosensitizers

In order to deliver the luminescent nanoparticles and the photosensitizers to the targeted tissue, they must be packaged together. In addition, the package must be compact in order to promote energy transfer from the nanoparticles to the photosensitizers thereby ensuring that efficient photoactivation can be accomplished. A typical mechanism for energy transfer is fluorescence resonance energy transfer (FRET). As used here, FRET refers to the transfer from the initially excited donor (the scintillation nanoparticle) to an acceptor (the photosensitizer). Efficient energy transfer must meet two requirements. First, the emission band of the donor must overlap effectively with the absorption band of the acceptor. Second, the donor and the acceptor must be close enough spatially to permit transfer. An important characteristic of FRET is that the transfer rate is highly dependent on the distance between the donor and receptor. The distance at which FRET is 50% efficient—called the Forster distance—is typically 2-10 nm. Generally, in order to have an efficient energy transfer, the distance between the donor and the acceptor should be less than 10 nm. This distance rule imposes limitations on the selection of the linkers and packaging options.

Photodynamic Therapy Agent Delivery

The packaged agents (scintillation nanoparticle and photosensitizer) must be delivered to targets, such as cancerous ovarian lesions. Selective targeting systems can be divided into two main types—namely passive and active targeting. Passive targeting is based on the phenomenon known as the enhanced permeability and retention (EPR) effect. EPR is a common effect in solid tumors. The enhanced vascular permeability of a solid tumor is important in the biology of the tumor, which greatly impacts the targeted delivery of macromolecular anticancer drugs.

In one embodiment of the presently disclosed and claimed SLPDT system, active targeting to selectively deliver agents by conjugates containing a receptor-targeting moiety is applied. (Reddi, 1997) The method is similar to antibody-antigen targeting and is, in some ways, better suited for PDT than passive targeting, particularly for ovarian tumors. Ovarian tumor targeting may be accomplished by attaching a tumor-specific ligand, such as folic acid, to the agents. Other receptor molecules may be used for other targets. Antibody-antigen targeting and avidin-biotin targeting may also be suitable for use in corresponding embodiments.

Folates are low molecular weight pterin-based vitamins required by eukaryotic cells for one-carbon metabolism and de novo nucleotide synthesis. The folate receptor is a glycosylphosphatidylinositol-anchored, high-affinity membrane folate binding protein that is over expressed in a wide variety of human tumors, including more than 90% of ovarian carcinomas (Wang and Low, 1998). On the other hand, normal tissue distribution of the folate receptor is highly restricted, making it a useful marker for targeted drug delivery to tumors. Folic acid, a high-affinity ligand for the folate receptor (K_{d}=˜10⁻¹⁰ M), retains its receptor binding property when covalently derivatized by its gamma-carboxyl group. Studies have shown that folate conjugates are taken into receptor-bearing tumor cells via folate receptor-mediated endocytosis (Wang et al, Journal of Controlled Release, 1998). Folate-conjugation, therefore, presents a useful method for receptor-mediated drug delivery into receptor-positive tumor cells.

Folic acid is potentially superior to antibodies as a targeting ligand because of its small size, lack of immunogenicity, ready availability, and simple, well-defined conjugation chemistry (Wang and Low, 1998). Folic acid is added to fortified foods and vitamin supplements. The covalent attachment of the vitamin folic acid to almost any molecule yields a conjugate that can be endocytosed into folate receptor-bearing cells. Because folate receptors are significantly overexpressed in the majority of human cancers, this methodology is currently being used for the selective delivery of imaging and therapeutic agents to tumor tissues.

In addition to cancer treatment, the presently disclosed and claimed SLPDT system can also be used to cure other diseases such as infectious diseases, sickle cell disease, stroke, Alzheimer's diseases, ulcers, Skin diseases, gun wounds, and many other diseases. For example, Helicobacter pylori is one bacterium that causes ulcers and has been implicated in stomach cancer. The presently disclosed and claimed SLPDT system can be used to kill H. pylori because H. pylori produces and accumulates porphyrins. By targeting scintillation nanoparticles to the H. pylori, a source of light can be provided to the porphyrins. Also, the presently claimed and disclosed invention can be used to treat airborne and food borne diseases that are caused by bacterial or viral infection. For examples, nanoparticle PDT can be used to kill E. Coli, Brucella and aerosol spores and to treat the diseases caused by these bacteria or viruses.

Methods and Description

The presently disclosed and claimed SLPDT system provides a new and efficient treatment modality for cancer by combining radiotherapy and photodynamic therapy. Radiation can be generated from a machine or a radioactive isotope that is attached or doped into the nanoparticles. As radiation is capable of penetrating into any position into the tissue, this offers a more efficient way for cancer treatment. Because no ex vivo light source is required, this modality is much more economical, simpler, more convenient and more effective than conventional PDT methods. The advantages of the present SLPDT system or modality are based on the following two novel and inventive concepts: (1) Compared to the light delivery from ex vitro, the light generated from the attached nanoparticles can activate the photosensitizers much more efficiently and therefore, the production of singlet oxygen is much higher; and (2) The combination of radiotherapy and SLPDT is more efficient for killing cancer cells than radiotherapy or SLPDT alone.

One methodology used to confirm the above-referenced concepts, includes the steps of: (1) Preparation of scintillation nanoparticles (for example, CaF₂:Mn²⁺); (2) Attachment of photosensitizers to the scintillation nanoparticles (CaF₂:Mn²⁺/Porphyrin); (3) Demonstration that energy transfer from the nanoparticles to the photosensitizers occurs and that subsequent singlet oxygen generation is a result of such energy transfer; (4) Demonstration that tumor destruction occurs via such a nanoparticle-based SLPDT compound; and (5) Evaluating and determining the in vivo toxicity of the CaF₂:Mn²⁺/Porphyrin agent. The procedures for other nanoparticles and nanoparticle SLPDT agents are the same or similar.

Alternatively, C₆₀, is contemplated for use as a photosensitizer. C₆₀ is a newly discovered promising photosensitizer for SLPDT that is available from several chemical companies, such as Aldrich. ZnO nanoparticles are used with C₆₀ because ZnO's emission spectrum is matched with the absorption spectrum of C₆₀ (i.e. 250-400 nm) (Kordatos et al, Chemical Physics, 2003, 293, 263), thus, energy transfer is efficient and practical for SLPDT.

Herein is demonstrated the preparation of CaF₂:Mn²⁺, CaF₂:Eu²⁺, ZnO, CdS, BaFBr:Eu²⁺, Mn²⁺, and CaPO₄:Mn²⁺ nanoparticles that have been coated with SiO₂ or ZnO nanoshells. Porphyrins or TiO₂ photosensitizers are then conjugated to these nanoparticles. Examples for making these nanoparticles are described below.

Preparation of CaF₂:Mn²⁺ Nanoparticles. The design and preparation of luminescent nanoparticles must take into account how to link these particles to photosensitizers such as porphyrins. Thiols such as L-cysteine and thioglycolic acid (TGA) are excellent stabilizers for making water-soluble nanoparticles, and porphyrins can be covalently linked via amine bonding. Thus, a linker with thiol groups on one end and amine groups at the other end would be an excellent linker to attach nanoparticles to photoporphyrins. L-cysteine is one of these bi-functional linkers. L-cysteine is used to stabilize CaF₂:Mn²⁺ nanoparticles. The Ca²⁺-containing solution is prepared by dissolving 0.555 g of Ca(NO₄)₂ and 0.085 g Mn(NO₄)₂ in 125 mL of water; 0.69 g of L-cysteine are added to the solution. The pH is adjusted to ˜10 by the addition of 0.1M NaOH. The solution is then purged with nitrogen for at least 30 minutes. Then, 0.1305 g NH₄F is dissolved in 5 mL deionized (DI) water and dropped into the solution slowly. After the completion of the reaction, a clear solution of CaF₂:Mn²⁺ nanocrystal nuclei is obtained. This solution is then refluxed at 100° C. to promote crystal growth. During the growth process, fractions with nanoparticles of different sizes are extracted and stored at 4° C. in the dark.

CaF₂:Eu²⁺, BaFBr:Eu²⁺, and CaF₂:Mn²⁺, Eu²⁺ nanoparticles are made using a similar recipe to that used for CaF₂:Mn²⁺ nanoparticles. A reducer, NaBH₄, was added to the solution during the chemical reaction to avoid the oxidation of Eu²⁺ and Mn²⁺. These nanoparticles have very strong luminescence, and an emission spectrum for CaF₂:Eu²⁺ nanoparticles is shown in FIG. 5. Results demonstrate that the co-doping of Eu²⁺ and Mn²⁺ in the nanoparticles creates the best fit for porphyrin-based SLPDT.

Preparation of ZnO Nanoparticles. ZnO Nanoparticles can be prepared by the reverse micelle method. Typically, two types of quaternary reverse micelles are made before the reaction. Both reverse micelle systems consist of surfactant cetyltrimethylammonium bromide (CTAB), cosurfactant n-hexanol, and oil phase n-heptane. The difference is that one contains zinc acetate aqueous solution, while the other contains hydroxide solution. The ratio of reverse micelle components can be varied according to different requirements of nanoparticle size. The two types of micelles are slowly mixed while stirring. An organosol of Zn(OH)₂ nanoparticles form inside the reverse micelle droplets. The white Zn(OH)₂ precipitates are then extracted from the reverse micelle system using 2-propanol. After several cycles of washing and centrifuging, the white Zn(OH)₂ nanoparticles are transferred to vacuum and dehydrated under low temperature to get ZnO nanoparticles. The nanoparticle sizes prepared by this method can be finely adjusted from several nanometers to tens of nanometers by altering the concentration ratio of water and CTAB.

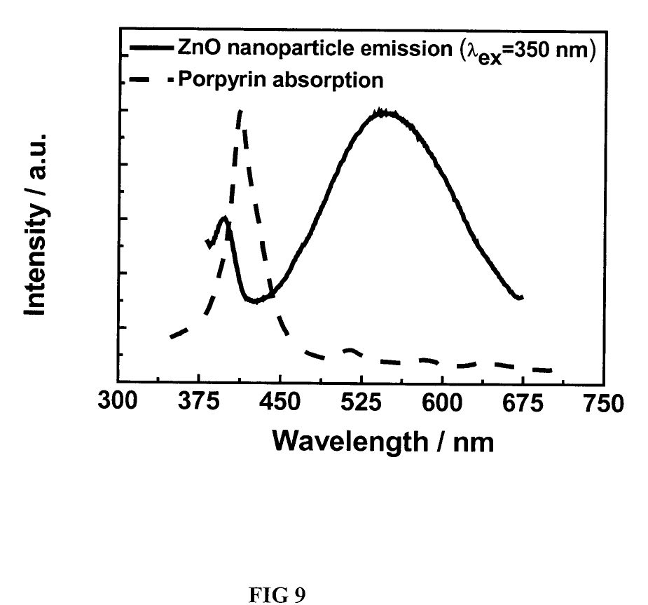

FIG. 9 shows the emission spectrum of ZnO nanoparticles and the optical absorption spectrum of a porphyrin. Obviously, the emission spectrum of ZnO nanoparticles overlaps effectively with the optical absorption spectrum of porphyrin. This indicates that there is efficient energy transfer from ZnO particles to porphyrins and the systems are efficient, therefore, for SLPDT.

Synthesis of CdS nanoparticles: The CdS nanoparticles were prepared via a reverse micelle method. This method allows the formation of uniform CdS nanoparticles with tunable sizes from 2 to 10 nm depending on the reaction parameters. The obtained CdS nanoparticles were well distributed in a micelle solution. To make them water soluble, the stabilizer exchange process was carried out after the nanoparticles formed. The CdS nanoparticles stabilized by thiol ligand were then extracted from the micelle to water. The preparation process is described below.

Two reverse micelles were prepared. Each of them contained three components: surfactant (CTAB), heptane, and n-hexanol. One contained a cadmium nitrate solution, and the other contained a sodium sulfide solution. The two reverse micelles were gradually mixed together under ultrasonication. As the solution gradually turned yellow, CdS nanoparticles were formed. The nanoparticle sizes and optical properties are strongly dependent on the preparation parameters—i.e., the molar ratio between water and CTAB (W), the molar ratio between n-hexanol and CTAB (P), and the molar concentration of CTAB ([CTAB]). In our preparation, the W, P, and [CTAB] were fixed at W=24.45, P=5.27 and [CTAB]=0.19 mol/L. The initial concentration of Cd²⁺ and S²⁻ was 8.24×10⁻⁴ mol/L.

The CdS nanoparticles were then transferred into a three neck flask, refluxing for 3 hours under nitrogen protection. This shifted the nanoparticle emission from green (575 nm) to blue (440 nm) to match the porphyrin absorption for energy transfer. The stabilizer exchange process was then carried out with the addition of an equal molar concentration of L-cysteine solution to replace CTAB. The water soluble CdS nanoparticles stabilized by thiol ligand were then extracted into the water phase. By discarding the organic phase, the water soluble CdS nanoparticles were obtained for bioconjugation with porphyrin.

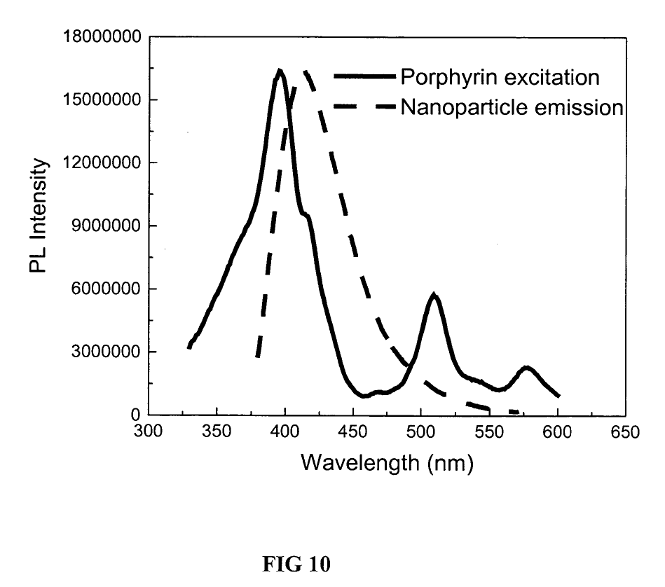

FIG. 10 shows the emission spectrum of CdS nanoparticles and the optical absorption spectrum of a porphyrin. The emission spectrum of CdS nanoparticles is overlapped effectively with the optical absorption spectrum of a porphyrin. This indicates that there is efficient energy transfer from CdS particles to porphyrins and the systems are efficient for SLPDT.

Nanoparticles Coated with ZnO or SiO₂ Shells

Coating with ZnO or SiO₂ improves the nanoparticle stability, enhances luminescence, reduces toxicity, and enhances SLPDT efficiency. Nanoparticle coating with ZnO or SiO₂ is a well-developed chemistry. Here is given one such known procedure for coating of CdS nanoparticles with SiO₂ shells.

A freshly prepared aqueous solution of 3-(mercaptopropyl) trimethoxysilane (MPS) (0.5 ml, 1 mM) is added to the CdS nanoparticle solution (50 ml) under vigorous stirring. The function of MPS is that its mercapto group can directly bond to the surface Cd sites of CdS, while leaving the silane groups pointing toward the solution. 2 ml of sodium silicate solution (pH 10.5) is added under vigorous stirring. The silicate ions bind with the silane groups of MPS. The resulting dispersion (pH ˜8.5) is allowed to stand for 5 days, so that the silica slowly polymerizes onto the particle surface. The dispersion is then transferred to ethanol, so that the excess dissolved silicate can precipitate out, increasing the silica shell thickness.

Conjugate Nanoparticles and Photosensitizers. The conjugation of photosensitizers to nanoparticles is a very important step in the SLPDT system presently disclosed and claimed. The coating of TiO₂ nanoparticles on scintillation or long-afterglow nanoparticles as described below is relatively easier than nanoparticle-porphyrin bioconjugation.

Nanoparticles/porphyrins conjugation. Molecules with bifuctional groups, such as L-cysteine are used to link the nanoparticles such as CaF₂:Mn²⁺, CaF₂:Eu²⁺, ZnO, TiO₂ and CdS to photosensitizers, such as porphyrin molecules. These molecules are also known as functional ligands. In this method, a calculated amount of the L-cysteine stabilized nanoparticles is dissolved in chloroform along with an excess amount of porphyrin. The mixture is stirred at room temperature for 24 hours. The excess unlinked porphyrin molecules are removed by dialysis. The procedure for linking porphyrins to other nanoparticles is similar.

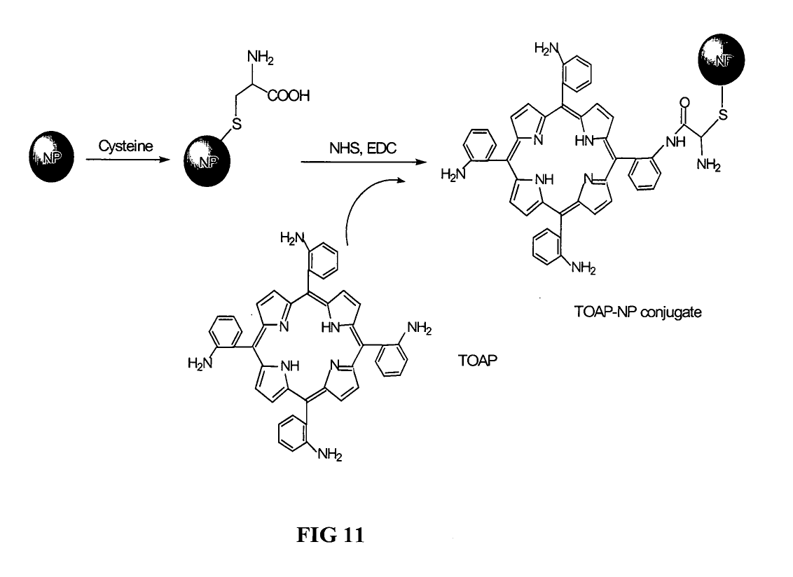

In one embodiment, one of the key design features is the attachment of a diamine linker of various lengths to the carboxylic acid group of the cysteine, via standard amino-protection and amide coupling conditions, to give a nanoparticle-cysteine-diamine intermediate (see FIG. 11). Biomolecules such as peptides modified with diamine increase their permeability through cell membranes. So the diamine motif can similarly benefit the delivery of the nanoparticle conjugate. The terminal amine group in the intermediate provides a chemical handle for the conjugation of TOAP. After deprotection of the t-butyloxycarbonyl group, the conjugate of NP-TOAP is formed.

Preparation of ZnO/TiO₂ Core/shell Nanostructure PDT agents. TiO₂ nanoparticle photosensitizers are coated to scintillation nanoparticles for the SLPDT system presently disclosed and claimed. Herein provided is one example for the synthesis of ZnO/TiO₂ core/shell nanoparticle PDT agents although one of ordinary skill in the art would appreciate that any such methodology could be used with the presently disclosed and claimed SLPDT system. The synthesis can be divided into two processes. The first process is to make size controlled ZnO nanoparticles as core materials via the reverse micelle method described above. The second process is to coat the ZnO nanoparticles with TiO₂ by controlled hydrolysis of ethanol solution of tetrabutyl titanate in the presence of ZnO nanoparticles and hydro-thermal treatment. The ZnO nanoparticles are well dispersed in PVP ethanol solution by ultrasonic treatment, and tetrabutyl titanate is dropped into ethanol with the help of ultrasonic agitation. Then DI water is added for further hydrolysis of tetrabutyl titanate with vibration. After being treated in a 50 ml Teflon autoclave at 105° C. for several hours, the solution is centrifuged at 6,000 rpm for 10 minutes. The precipitates are washed several times with ethanol and DI water. Purified ZnO/TiO₂ core/shell nanoparticles are obtained after being dried in a vacuum. The nanopowders can be dissolved into water easily by surface modification. In a similar way, CaF₂:Eu²⁺/SiO₂/TiO₂ PDT agents can also be fabricated.

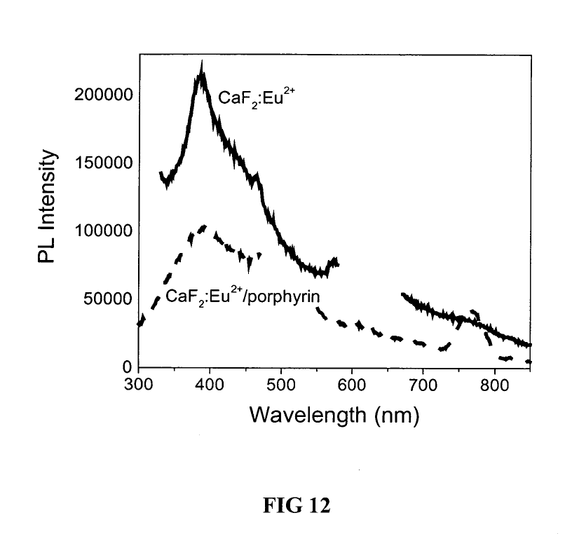

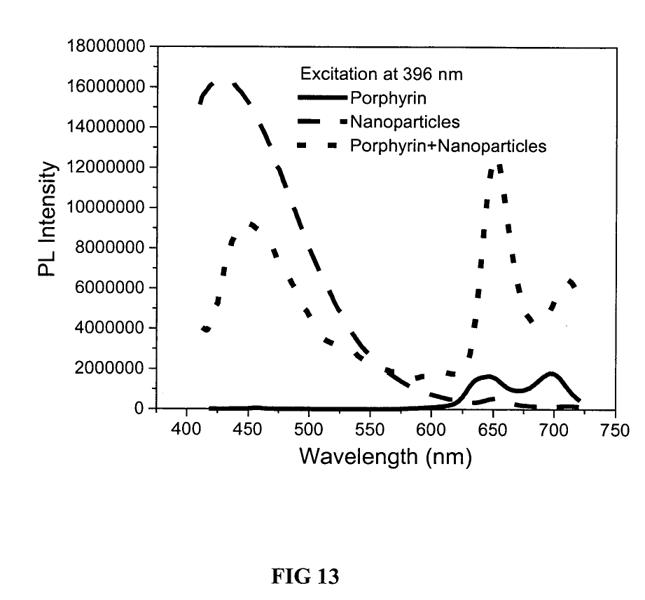

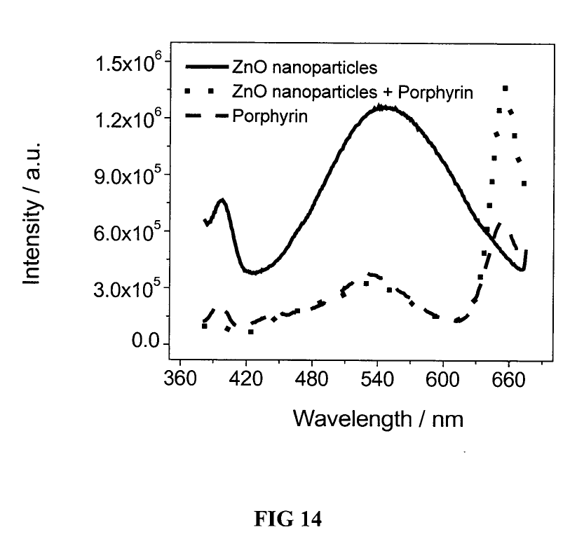

Energy Transfer and Singlet Oxygen Generation. The energy transfer from the nanoparticles to the photosensitizers and the subsequent generation of singlet oxygen are needed for effective cancer treatment. As an example, the details of how to realize and observe energy transfer from scintillation or long-afterglow nanoparticles to photosensitizers and the generation of singlet oxygen are herein set forth. Efficient energy transfer from the scintillation nanoparticles to the photosensitizers is prerequisite for the generation of singlet oxygen for PDT. Two methods can be used to study and measure the energy transfer. With luminescence quenching, the luminescence efficiency or intensity of the scintillation nanoparticle is quenched when the photosensitizers are attached to the particles as energy transfers from the scintillation nanoparticles to the photosensitizers. This is a simple and direct method to study energy transfer between nanoparticles or between fluorophors. Results of such tests are shown in FIGS. 12-14 for CaF2:Eu²⁺/porphyrin, CdS/porphyrin and ZnO/porphyrin systems. After bio-conjugation, the emission of the nanoparticles is quenched but the emission from the porphyrin is enhanced in intensity. This indicates that the energy transfer from the nanoparticles to the porphyrins is accomplished.

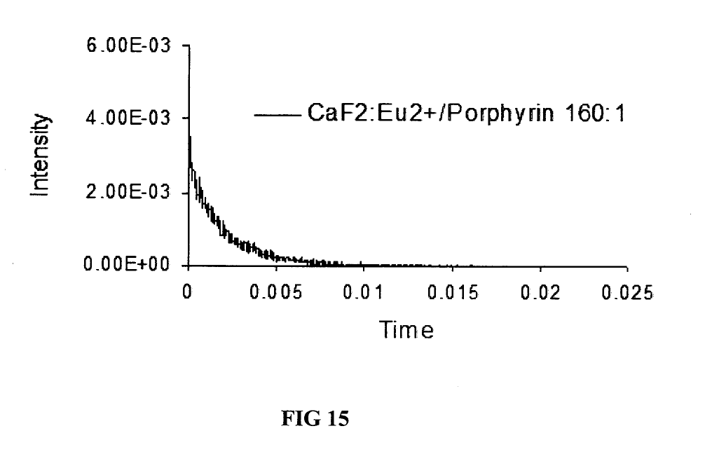

A complimentary and reliable method is called lifetime quenching, in which the luminescence decay lifetime of the scintillation particles is shortened if there is energy transfer to the photosensitizers. Because the shortening rate is related to the energy transfer rate, lifetime measurements can provide good estimates of the energy transfer rate. The decay lifetime of Eu²⁺ in CaF₂:Eu²⁺-porphyrin is about 620 ns (FIG. 15), which is about 180 ns shorter than the decay lifetime of Eu²⁺. This is strong evidence that there is energy transfer from CaF₂:Eu²⁺ nanoparticles to porphyrins.

The generation of singlet oxygen from the self-lighting PDT agents is also a prerequisite for effective cancer treatment. Singlet oxygen has a very short lifetime, 10-100 μs in organic solvents and several microseconds in water. Therefore, the measurement of singlet oxygen is a difficult task. Fortunately, singlet oxygen emits light in the near-infrared range at 1270 nm when it returns to triplet oxygen, which is the ground state of oxygen. Singlet oxygen emission provides a method for measuring singlet oxygen concentration.

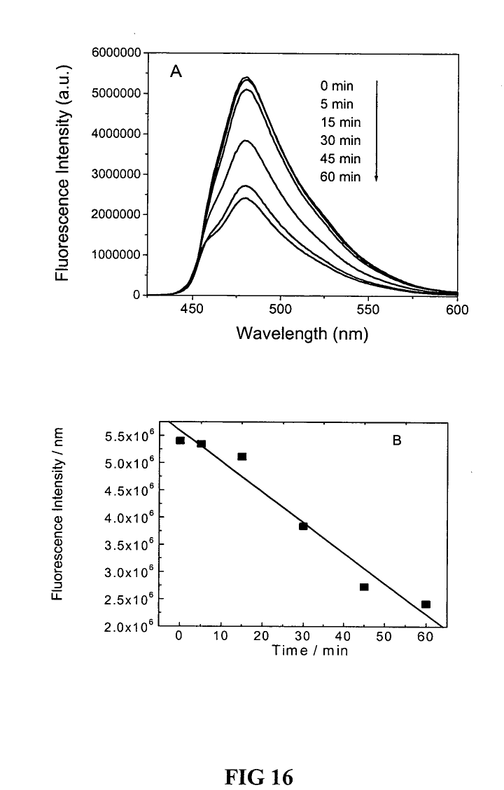

Singlet oxygen also can be detected by chemical quenchers (Belfield, et al, 2005). 1,3-diphenylisobenzofuran (DPBF) was applied to monitor and measure the generation of singlet oxygen in the presently claimed and disclosed SLPDT systems. The oxidation of DPBF with singlet oxygen produces a non-fluorescent product. Thus, by measuring the decrease in the fluorescence intensity of DPBF, the generation and the amounts of singlet oxygen can be measured. Typical and exemplary results showing the production of singlet oxygen are given in FIG. 16.

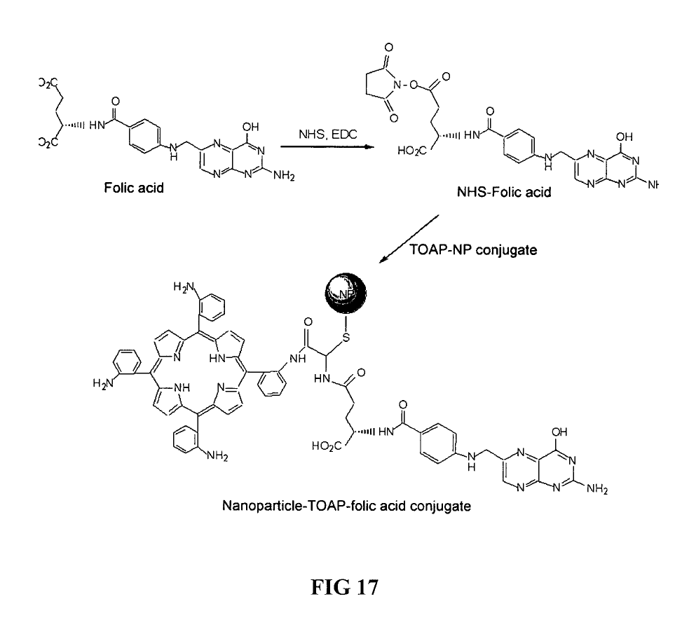

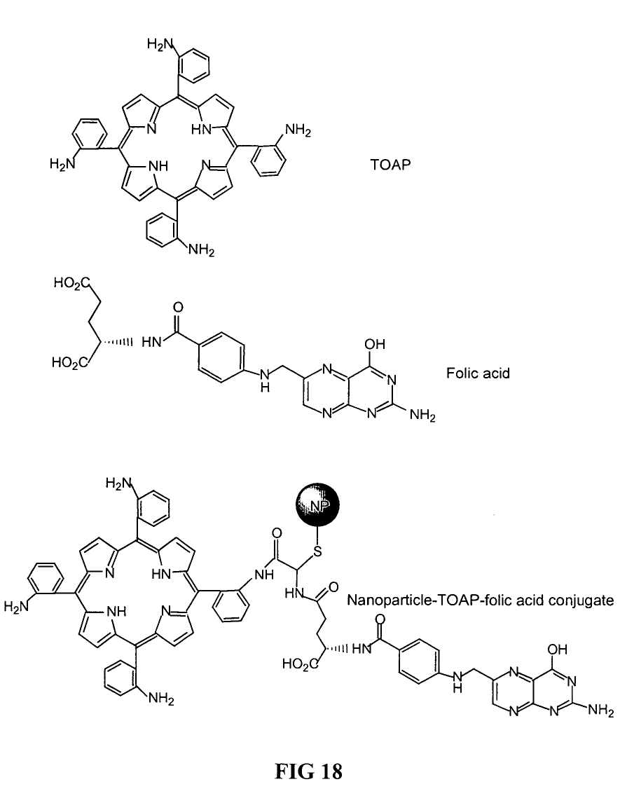

Coat and/or Encapsulate Conjugates with Folic Acid for Tumor Targeting. As described hereinabove, folic acid can target the folate receptor in a highly specific manner. In order to take advantage of this specific binding affinity to ovarian tumor, a conjugate that consists of folic acid, verteporfin, and nanoparticles was synthesized. The conjugate was designed carefully so as to avoid affecting the functionality of the PDT agents and the binding affinity of folic acid for ovarian tumors. Folic acid retains its receptor binding properties when derivatized via its γ-carboxyl. So, the γ-carboxyl group of folic acid was chosen as the point of connection. The selective activation of γ-carboxylic acid to N-hydroxysuccinimide ester, followed by the amide formation with the terminal-amino group of the nanoparticle-photosensitizer conjugate completes the synthesis of the nanoparticle-photosensitizer-folic acid conjugate. These processes are illustrated in FIGS. 17-18, respectively. FIG. 17 is a schematic illustration of chemical process used for making TOAP-nanoparticle-folic acid conjugates and FIG. 18 is a schematic illustration showing the methodology of linking TOAP and folic acid to a luminescent nanoparticle. For simplicity, only one TOAP and one folic acid molecule per nanoparticle is shown. The carboxylic group of the cysteine is used to connect with amino group of TOAP, while the amino group of the cysteine is linked to with the carboxylic group of the folic acid by an amide bond.

To further improve the solubility and circulation longevity, folic acid-coated liposomes may be used to deliver the nanoparticle PDT agent to tumors and specifically ovarian tumors. In recent decades, liposomes have been used in numerous applications as a delivery vehicle for therapeutic and diagnostic agents. Liposomes achieved such popularity because of their amphiphilic nature and compatibility to biological systems. Three main challenges remain to fully utilize liposomes as a delivery tool: (1) economical and easy preparation of functionalized liposomes, (2) precise control of liposome morphologies, and (3) improved target-specificity. In order to increase the target-specificity of the liposome system, the surface of the liposome is chemically modified. Antibody-antigen targeting, avidin-biotin targeting, and specific receptor molecules may all be used. In one embodiment, the surface of the liposome is modified with folic acid, a well-known ligand specific to the folate receptor. The folic acid is attached to the liposome by a covalent bond, and functions as a targeting recognition unit to tumor cells.

There are two general approaches to functionalizing the surface of a liposome: (1) make and purify functionalized phospholipid monomers and assemble them into a modified liposome, and (2) make modifications directly on the native liposome bilayer. The first strategy is more practical and preferred because of the ability to precisely control the chemical composition of the liposome surface. In addition, all chemical reagents are readily available from commercial sources. Therefore, the γ-carboxylate of folic acid is activated to its succinimide ester and then forms a covalent amide bond with the terminal amino group in phosphatidyl ethanolamine (PE), the monomer that forms the liposome. Modification of the γ-carboxylate of folic acid (FA) does not effect its affinity to the folate receptor.

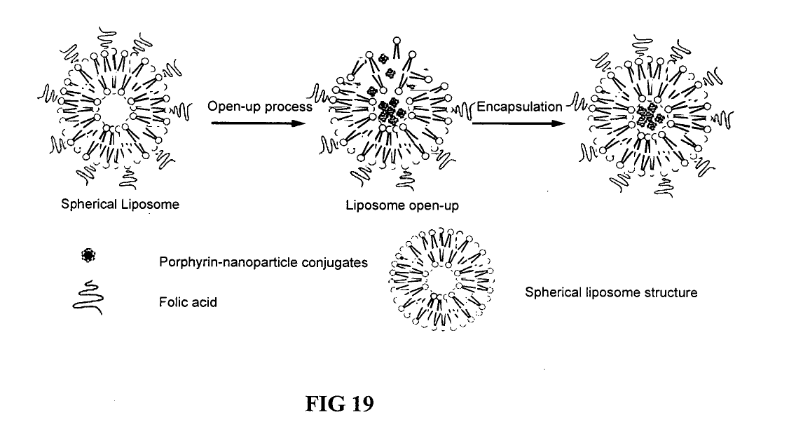

The PE-FA conjugate monomer can then be assembled to form a liposome using a standard protocol, which normally involves dissolving the PE-FA monomer in organic solvent and then dispersion in an aqueous solution to induce the formation of liposomes. Encapsulation of the nanoparticle-photosensitizer takes advantage of the morphological changes of liposomes induced by the interactions between the liposome membrane and talin, a cytoskeletal protein. Talin can bind to the membrane directly and promote actin polymerization. When added to a liposome solution, it induces a stable hole and transforms the liposome from a spherical structure to an open, cup shape. This morphological change is reversible, however, by dilution of talin (Saitoh, et al, 1998). This reversible open-close process is useful to encapsulate the nanoparticle-photosensitizer conjugate. Once the liposome opens at high concentration of talin, the nanoparticle-photosensitizer conjugate is added to the liposome solution and mixed thoroughly. After the nanoparticle-photosensitizers enter the opened liposomes, the liposomes are induced to close and encapsulate the nanoparticle-photosensitizer conjugates by gradually decreasing the talin concentration (FIG. 19). The separation of the liposome-encapsulated nanoparticle-photosensitizers from the free nanoparticle-photosensitizers can be further achieved via an ion exchange method, based on the affinity of an exchange resin for charged nanoparticle-photosensitizers and the repulsion by the same resin of oppositely charged liposome encapsulated nanoparticle-photosensitizers.

In vivo Test of Self-Lighting Photodynamic Therapy. Self-lighting PDT does kill cancer cells. The particles have been used in tissue culture experiments showing that the use of self-lighting PDT does, in fact, kill cancerous cells. Cell survival experiments are conducted wherein varying quantities of coated particles are incubated with human ovarian cancer cells (OVG1) and, as a function of time after incubation, the cells are treated with varying doses of ionizing radiation (X-ray source). The cells are then washed, plated, and, 7-10 days after irradiation, are counted after staining with methylene blue. This type of in vitro assay tests the reproductive integrity of the cancer cells. Appropriate controls are used, and the efficiency of tumor killing is recorded.

In a similar fashion, athymic or SCID mice that are immunologically compromised are injected (a subcutaneous injection of 2×10⁵ cells suspended in 0.1 mL phosphate buffered saline are injected into the right hind leg using a 27-gauge needle) with OVG1 cells. The inoculum is allowed to grow for 10-15 days at which time the tumor is 1+/−0.1 cm in diameter, whereupon varying doses of X-ray are delivered exclusively to the right hind leg; the rest of the mouse is shielded from radiation by lead affixed to specially designed acrylate jigs. There are three control groups: one received no nanoparticles but is irradiated, one received nanoparticles but no irradiation, and one received nanoparticles and irradiation. There are also several radiation doses. In particular, the dose is one single fraction of 10, 15, 20, and 25 Gy. Efficacy is established by recording the size of the tumor every day and comparing the growth of tumors that have not been treated to the growth rates of tumors treated by either radiation or nanoparticles alone or in combination with nanoparticles plus irradiation. Efficacy is then determined by noting how much more effective the combination treatment is when compared to the growth delay induced by radiation alone or nanoparticles alone. There are five animals per group.

One of the challenging issues is to determine how much radiation dose is sufficient to generate enough light for photodynamic therapy. When patients receive radiation for the treatment of their tumors, the normal daily dose is about 2 Gy given daily (5 days a week) for 5-7 weeks. The scintillation luminescence efficiency of our nanoparticles is around 50-60%. In each measurement, we need 0.025 Gy of X-ray dose for a spectral measurement. This equals a radiation duration of 0.5 seconds using an Oxford Instruments XTF5011 X-ray tube with a tungsten target operating at 25 kV and 0.5 mA. The dose is equivalent to 2 Gy for 40 seconds, which will kill all the tumor cells, leaving nothing for PDT treatment. The challenge is to limit X-ray dosage while generating enough light for effective PDT. To limit the X-ray dosage, the use of persistent luminescence nanoparticles is contemplated for use with the presently disclosed and claimed SLPDT system. These nanoparticles have a long-lasting afterglow that is visible up to two hours after the excitation. The afterglow can be charged by irradiation for less than 5 seconds. In this case, even if the X-ray is off, the PDT is still active because of the long afterglow from the nanoparticles. This provides an efficient, simple, convenient, and economical light source for PDT treatment.

### EXAMPLE 1 STUDIES FOR CYTOTOXICITY AND CANCER CELL KILLINGS

For CaF₂:Eu²⁺, no dark toxicity was observed which is good for in vivo applications. However, CdTe nanoparticles did elicit concentration-dependent cytotoxicity. To reveal the origin of the toxicity, red nanoparticles were purified by precipitation and then dissolved back to aqueous solution. After purification, which putatively removed the free Cd²⁺ ions, the toxicity was greatly reduced. This indicates that the toxicity is probably due to free Cd²⁺ ions in the solution. Furthermore, purification and coating with silica (to prevent Cd²⁺ from leakage) may lead to non-toxic or less-toxic nanoparticles. ZnO nanoparticles have greater toxicity than bulk ZnO and were capable of killing liver tumor cells.

Male Sprague Dawley rats (100-120 g body weight) were given red CdTe nanoparticles (1 ml/kg) by intravenous administration via the tail vein. Control rats (n=3) received saline only (1 ml/kg). Locomotor activity was measured for 2 hours (1000-1200 hours) on the day prior to dosing, just after dosing, and then 24 hours after dosing. After motor activity measurements on day three, the rats were sacrificed and blood samples collected in heparinized vials for separation of plasma. Spleen, brain, lung, liver, heart, and kidney were collected into formalin for histopathology. Blood urea nitrogen (BUN) and creatinine were measured in plasma samples by the Pathology Department at Boren Veterinary Medicine Teaching Hospital, Oklahoma State University.

The results indicate that the motor activity was significantly altered by the nanoparticle treatment (a significant reduction in rearing for two hours after dosing, and a significant increase in both rearing and ambulation 24 hours after dosing). As motor activity is a typical measurement of nervous system function, this suggests a neurotoxic effect of the nanoparticle exposure. Furthermore, while neither BUN nor creatinine were significantly affected, there was a significant change in the BUN/creatinine ratio with nanoparticle dosing. This may indicate a change in renal function can occur following red CdTe nanoparticle systemic administration.

ZnO/porphyrin and CdS/porphyrin conjugates were also tested for tumor cell and bacterial killing. Results indicate that these nanoparticle-porphyrin systems can kill tumor cells and bacteria (Brucella) efficiently under ultraviolet light stimulation which further demonstrates that these systems are potentially efficient PDT agents for cancer treatment, bacteria or virus destruction.

### REFERENCES

1) J. W. Arbogast, A. P. Darmanyan et al, Photophysical properties of C₆₀, J. Phys. Chem. 1991, 95: 11
2) Wei Chen, Jiaqing Song, Mianzeng Su, Electron Migration in BaFCl:Eu²⁺ Phosphors, Journal of Applied Physics, 1997, 81(7):3170-3174
3) K. D. Belfield, C. C. Corredor, A. R. Morales, M. A. Dessources, and F. E. Hernandez, Journal of Fluorescence, 2005, DOI: 10.1007/s10895-005-0008-1
4) D. L. Dexter, A theory of sensitized luminescence in solids, J. Chem. Phys. 21, 836 (1953).
5) K. Kordatos, T. Da Ros, M. Prato, R. V. Bensasson, and S. Leach, Absorption spectra of the mono-adduct and eight bis-adduct regioisomers of pyrrolidine derivatives of C60, Chemical Physics, 2003, 293: 263-280.
6) I. Macdonald, T. Dougherty, Basic principles of PDT, J. Porphyrins and Pthalocyanines, 2001, 5: 105
7) H. Maeda, SMANCS and polymer-conjugated macromolecular drugs: advantages in cancer chemotherapy, Adv. Drug Deliv. Rev. 1991, 6:181
8) E. Reddi, Role of delivery vehicles for photosensitizers in the photodynamic therapy of tumors, J. Photochem. Photobiol. 1997, 37: 189
9) Akihiko Saitoh, Kingo Takiguchi, Yohko Tanaka, and Hirokazu Hotani, Opening-up of liposomal membranes by talin, Proc. Natl. Acad. Sci. USA, 1998, 95, 1026-1031.
11) T. Takagahara, effect of dielectric confinement and electron-hole exchange interaction on excitonic states in semiconductor quantum dots, Phys. Rev. B., 1993, 47: 4569
12) S. Wang, R. Gao, F. Zhou and M. Selke, Nanomaterials and singlet oxygen photosensitizers: potential applications in photodynamic therapy, J. Materials Chemistry, 2004, 14: 487
13) S. Wang, P. S. Low, Folate-mediated targeting of antineoplastic drugs, imaging agents, and nucleic acids to cancer cells, J. Controlled Release, 1998, 53: 39
14) S. Xu, J, Shen, S. Chen, M. Zhang, and T. Shen, Active oxygen species generation if the system of TiO₂ colloid sensitized by hypocrillin B., J. Photochem. Photobiol. B: Biology, 2002, 67:64

What is claimed is:

1. A method for photodynamic therapy comprising the steps of:
providing at least one luminescent nanoparticle;
providing at least one photosensitizer, wherein the at least one photosensitizer is functionally associated with the at least one luminescent nanoparticle; and
providing an excitation source, wherein the excitation source is capable of exciting the at least one luminescent nanoparticle to thereby excite the at least one photosensitizer to provide the photodynamic therapy.

2. The method of claim 1, wherein the luminescence of the at least one luminescent nanoparticle is selected from the group consisting of scintillation luminescence, persistent luminescence, afterglow, thermoluminescence, magnetoluminescence, phosphorescence, photostimulated luminescence, and bioluminescence.

3. The method of claim 1, wherein the at least one luminescent nanoparticle is selected from the group consisting of semiconductor nanoparticles, insulator nanoparticles, doped nanoparticles, ceramic nanoparticles, metallic nanoparticles, organic nanoparticles, inorganic nanoparticles, core-shell nanoparticles, size confined nanoparticles, dielectric confined nanoparticles, size and dielectric doubly confined nanoparticles, and combinations thereof.

4. The method of claim 1 wherein the at least one luminescent nanoparticle has a diameter from about 0.1 nm to about 5000 nm.

5. The method of claim 1, wherein the at least one luminescent nanoparticle is selected from the group consisting of CaF₂:Mn²⁺, CaF₂:Eu²⁺, CaF₂:Ce³⁺, BaFBr:Eu²⁺, BaFBr:Mn²⁺, CaPO₄:Mn²⁺, CaPO₄:Eu²⁺, ZnS, ZnO, CdS, CdSe, CdTe, TiO₂ nanoparticles and combinations thereof.

6. The method of claim 1, wherein the at least one photosensitizer is selected from the group consisting of organic dyes, porphyrins and their derivatives, flavins, organometallic species, inorganic compounds, fullerenes, semiconductor nanoparticle photosensitizers, and combinations thereof.

7. The method of claim 1, wherein the at least one photosensitizer is a porphyrin.

8. The method of claim 7, wherein the at least one photosensitizer is selected from the group consisting of haematoporphyrin, verteporfin, tetrakis(o-aminophenyl)porphyrin, and combinations thereof.

9. The method of claim 1, wherein the at least one photosensitizer is selected from the group consisting of ZnO nanoparticles, Si nanoparticles, TiO₂ nanoparticles and combinations thereof.

10. The method of claim 1, wherein the at least one nanoparticle and the at least one photosensitizer are operably associated with one another by a functional ligand.

11. The method of claim 10, wherein the functional ligand is cysteine.

12. The method of claim 1, wherein the at least one nanoparticle and the at least one photosensitizer are operably associated with one another by electrostatic interaction.

13. The method of claim 1, wherein the at least one nanoparticle and the photosensitizer are operably associated with one another by coating the at least one photosensitizer on the surface of the at least one nanoparticle.

14. The method of claim 13, wherein the at least one photosensitizer is selected from the group consisting of TiO₂, ZnO and combinations thereof.

15. The method of claim 13, wherein the at least one nanoparticle is selected from the group consisting of CaF₂:Eu²⁺, ZnO and combinations thereof.

16. The method of claim 1, wherein the excitation source is an ionizing radiation source.

17. The method of claim 16, wherein the radiation source produces radiation selected from the group consisting of X-rays, alpha-particles, beta-particles, neutrons, gamma rays and combinations thereof.

18. The method of claim 16, wherein the radiation source is at least one radioactive atom doped in or bound to the at least one luminescent nanoparticle.

19. The method of claim 16, wherein the radiation source is capable of at least two functions comprising radiation therapy and excitation of the at least one luminescent nanoparticle, wherein the excited luminescent nanoparticle is capable of exciting the at least one photosensitizer and thereby provide the photodynamic therapy.

20. The method of claim 1, wherein the excitation source is heat.

21. The method of claim 20, wherein the heat is generated by a method selected from the group consisting of infrared light, a magnetic field and combinations thereof.

22. The method of claim 1, wherein the method for photodynamic therapy is used for the photodynamic treatment of cancer or a tumor in a patient.

23. The method of claim 22, wherein the tumor is a bladder tumor.

24. The method of claim 22, wherein the cancer is selected from the group consisting of breast cancer, prostate cancer, skin cancer, ovarian cancer and combinations thereof.

25. The method of claim 1, wherein the method for photodynamic therapy is used for the photodynamic treatment of an infectious disease in a patient.

26. The method of claim 25, wherein the infectious disease is caused by a bacteria or a virus.

27. The method of claim 26, wherein the bacteria is E. coli.

28. The method of claim 26, wherein the virus is selected from the group consisting of an influenza virus, a severe acute respiratory syndrome (SARS) virus and combinations thereof.

29. The method of claim 1, further including the step of providing targeting of the least one luminescent nanoparticle that is functionally associated with the at least one photosensitizer.

30. The method of claim 29, wherein the targeting is provided by a method selected from the group consisting of antibody-antigen targeting, receptor targeting and combinations thereof.

31. The method of claim 29, wherein the targeting is provided by the conjugation of folic acid to the at least one luminescent nanoparticle.

32. The method of claim 29, wherein the targeting is provided by the encapsulation of the at least one luminescent nanoparticle functionally associated with the at least one photosensitizer in at least one liposome, wherein said liposome has a functionalized surface that acts as a receptor.

33. A method for photodynamic therapy comprising the steps of:
providing at least one luminescent photosensitizer nanoparticle, and
providing an ionizing radiation source, wherein the ionizing radiation source is capable of exciting the at least one luminescent photosensitizer nanoparticle to provide photodynamic therapy.

34. The method of claim 33, wherein the at least one luminescent photosensitizer nanoparticle is selected from the group consisting of ZnO nanoparticles, Si nanoparticles, TiO₂ nanoparticles and combinations thereof.
